(19) United States
(12) Patent Application Publication
(10) Pub. No.: US 2020/0197552 A1
(43) Pub. Date: Jun. 25, 2020

(54) Special sterilization cabinet for wooden cutting boards

(71) Applicant: Wangyan Qian, Shenzhen City (CN)

(72) Inventor: Wangyan Qian, Shenzhen City (CN)

(21) Appl. No.: 16/808,418

(22) Filed: Mar. 4, 2020

(30) Foreign Application Priority Data
Nov. 12, 2019 (CN) 2019111024074

(51) Int. Cl. A61L 2/10; B24B 41/00; B24B 41/02; B24B 7/04

(52) U.S. Cl. CPC A61L 2/10; B24B 41/005; A47J 47/005; B24B 7/04; A61L 2202/17; B24B 41/02

(57) ABSTRACT

The invention discloses a special disinfection cabinet for wooden cutting boards. The disinfection cabinet includes a disinfection cabinet. The disinfection cabinet is provided with a disinfection cavity which is opened forward. The disinfection cavity is provided with a support plate. The cutting board is placed on the left end face of the support plate, the cutting board is clamped by the clamping mechanism, and a pushing mechanism is provided on the right side of the support plate. Before disinfecting the cutting board, the invention first polishes the surface of the cutting board Grind off the surface of the cutting board, so that most mold can be removed, and the knife marks on the surface of the polished cutting board can also be removed to prevent bacteria from accumulating and breeding. Then rinse the cutting board and use ultraviolet radiation to Compared with the traditional disinfection method, the disinfection effect is better.

### CROSS-REFERENCES TO RELATED APPLICATIONS

The present application claims priority from Chinese application No. 2019111024074 filed on Sep. 19, 2019 which is hereby incorporated by reference in its entirety.

### TECHNICAL FIELD

The invention relates to the field of sterilization technology, in particular to a special sterilization cabinet for wooden cutting boards.

### BACKGROUND OF THE INVENTION

The chopping board refers to the utensils that are placed underneath when slicing, cutting, chopping, or smashing things. It is generally used for cooking. The chopping board is generally made of wood. In long-term use, the chopping board is in a wet state for a long time. Under the action of wood materials, the cutting board will mold, and due to prolonged use, there will be more dents on the surface of the cutting board, which will breed more bacteria, including Aspergillus flavus, etc. Aspergillus flavus is highly toxic. It is dangerous to eat with care. At this stage, the disinfection of wooden cutting boards is generally boiling water, sunlight, etc., but it is difficult to completely disinfect. To solve the above problems, the present invention is proposed.

### BRIEF SUMMARY OF THE INVENTION

The technical problem to be solved by the present invention is to provide a special disinfection cabinet for wooden cutting boards, which overcomes the above problems.

The present invention is achieved by the following technical solutions.

A special disinfection cabinet for wooden cutting boards includes a disinfection cabinet, the disinfection cabinet is provided with a disinfection cavity opening forward, the disinfection cavity is provided with a support plate, and the support plate is provided with a clamping mechanism for placing the anvil plate. The left end face of the support plate is used to clamp the cutting board by the clamping mechanism. A push mechanism is provided on the right side of the support plate. After the push mechanism is activated, the support plate is moved to the left, and the disinfection chamber A first electric guide rail is fixed in the top wall, an upper mounting block is installed on the lower side of the first electric guide rail, a lower mounting block that can slide back and forth is provided on the bottom wall of the disinfection chamber, and a power is provided in the lower mounting block. A motor, a rotary shaft is dynamically connected to the upper side of the power motor, the upper end of the rotary shaft is rotatably connected to the lower end of the upper mounting block, a grinding wheel is fixed on the rotary shaft, and a positioning sensor is provided on the rear wall of the disinfection chamber After the positioning sensor detects that the cutting board moves a certain distance to the left, the pushing mechanism stops, the first electric guide rail starts to move the grinding wheel forward, and the left end surface of the cutting board is polished away by a certain thickness, From To realize the removal of mildew and knife marks, a cleaning mechanism located on the left side of the grinding wheel is provided in the disinfection chamber. After the grinding is completed, the cleaning mechanism is opened to rinse the grinding wheel and the cutting board, and the disinfection chamber An ultraviolet disinfection mechanism is provided, and the cleaned cutting board is irradiated and disinfected after the ultraviolet disinfection mechanism is turned on.

Further, the cleaning mechanism includes a fixing plate fixedly disposed on the left wall of the disinfection chamber, a nozzle is installed on the right end surface of the fixing plate, and a water inlet pipe extends into the disinfection chamber to communicate with the nozzle, and the disinfection chamber The bottom wall is provided with a drain pipe extending outside the disinfection cavity.

Further, the ultraviolet disinfection mechanism includes ultraviolet lamps that are symmetrically fixed up and down on the upper and lower inner walls of the disinfection cavity. A reflector is provided on the left side of the ultraviolet lamp. After the ultraviolet lamp is activated, the light is reflected by the reflector to the The left end surface of the cutting board is described.

Further, an electric push rod is fixed on the rear wall of the disinfection cavity, a connecting rod is fixed on the front end of the push rod on the front side of the electric push rod, and a cover plate is symmetrically arranged on the front surface of the connecting rod. The reflecting surface of the reflecting mirror is shielded, thereby avoiding contamination of the reflecting surface of the reflecting mirror during grinding and cleaning.

Further, the pushing mechanism includes a rotary motor provided in the right wall of the disinfection chamber, a left side of the rotary motor is power-connected with a threaded rod, and a threaded sleeve threadedly connected to the threaded rod is provided in the disinfection chamber. The left end of the threaded sleeve is fixedly connected to the support plate, the threaded sleeve is provided with a guide groove opening upward, and the sterilization chamber is provided with a limiting block slidingly installed in the guide groove. After the rotary electric machine is started, the threaded sleeve moves to the left under the restriction of the restriction block.

Further, a second electric guide rail is fixed in the inner wall of the disinfection chamber, and a middle rod is installed on the left side of the second electric guide rail, and the middle rod is fixedly connected to the restriction block. After the cutting board is rinsed, When the second electric guide rail is activated, the restriction block rises and disengages from the guide groove, and when the rotary motor is activated, the threaded rod drives the threaded sleeve to rotate, and the cutting board is dried.

Further, the clamping mechanism includes a lower clamping plate fixedly disposed on the left end surface of the support plate, and a third electric guide rail located at the upper side of the lower clamping plate is fixedly disposed on the left end surface of the support plate. An upper clamping plate is installed on the left side of the third electric guide rail. The cutting board is placed between the lower clamping plate and the upper clamping plate, and the third electric guide rail is activated to lower the upper clamping plate. The cutting board is clamped, and the length of the lower clamping board and the upper clamping board is shorter than the thickness of the cutting board.

Further, a dust suction mechanism at the rear side is installed between the upper mounting block and the lower mounting block, and a mounting shaft is rotatably provided between the upper mounting block and the lower mounting block, and the mounting A torsion spring is connected between the shaft and the top wall of the upper mounting block, and a shielding plate located on the rear side of the dust suction mechanism is fixed on the mounting shaft.

Further, a front door of the sterilization cabinet is hingedly installed with a cabinet door for closing the opening on the front side of the sterilization chamber, and an indicator light is installed on the front surface of the sterilization cabinet, and the indicator light can be illuminated during and after disinfection. Different colored lights.

Beneficial effects of the present invention: Before sterilizing the cutting board, the present invention first grinds the surface of the cutting board and grinds off the surface of the cutting board, so that most mold can be removed, and knife marks on the surface of the polished cutting board etc. It can also be removed to prevent bacteria from accumulating and breeding. Then, after rinsing the cutting board, it is sterilized with ultraviolet radiation. Compared with traditional disinfection methods, the disinfection effect is better.

### BRIEF DESCRIPTION OF THE DRAWINGS

In order to more clearly explain the embodiments of the invention or the technical solutions in the prior art, the drawings used in the description of the embodiments or the prior art will be briefly introduced below. Obviously, the drawings in the following description are merely For some embodiments of the invention, for those of ordinary skill in the art, other drawings can be obtained based on these drawings without paying creative labor.

FIG. 1 is a schematic diagram of the overall structure of the present invention;

FIG. 2 is a schematic cross-sectional structure diagram of FIG. 1;

FIG. 3 is a schematic structural view at A-A in FIG. 2;

FIG. 4 is a schematic structural view at B-B in FIG. 2..

### DETAILED DESCRIPTION OF THE INVENTION

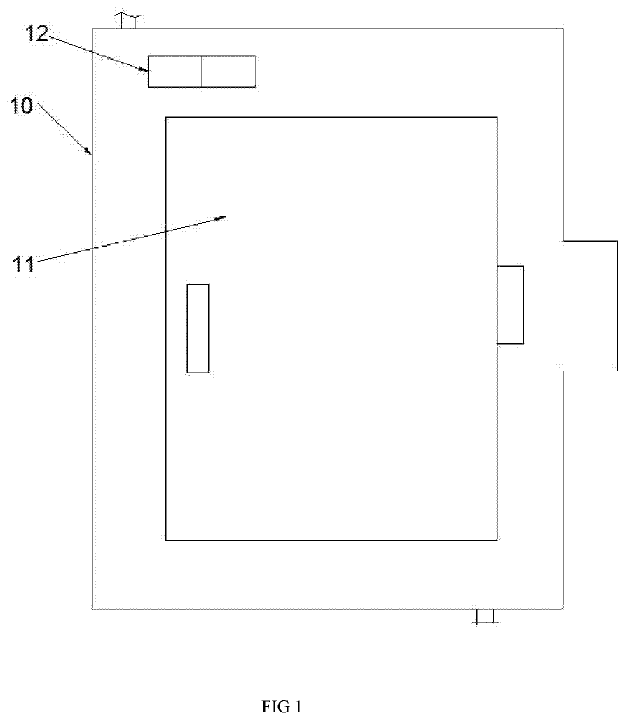

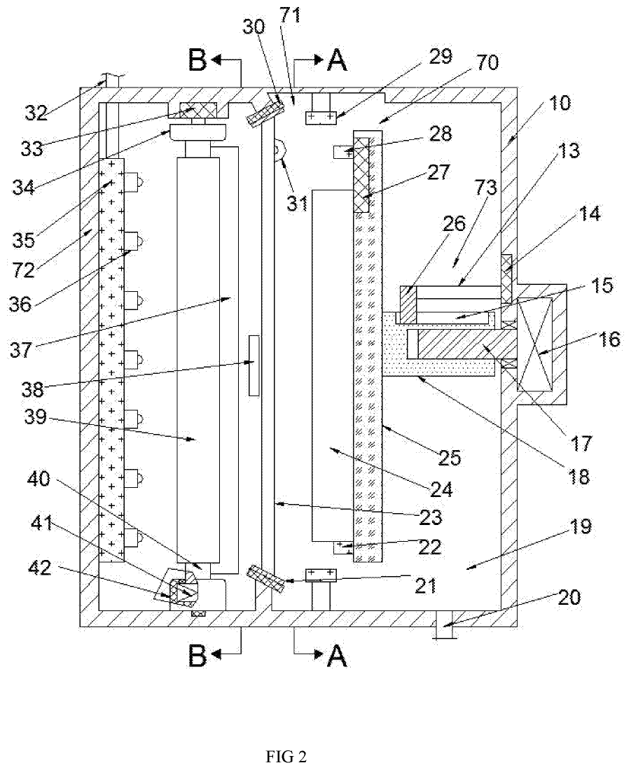

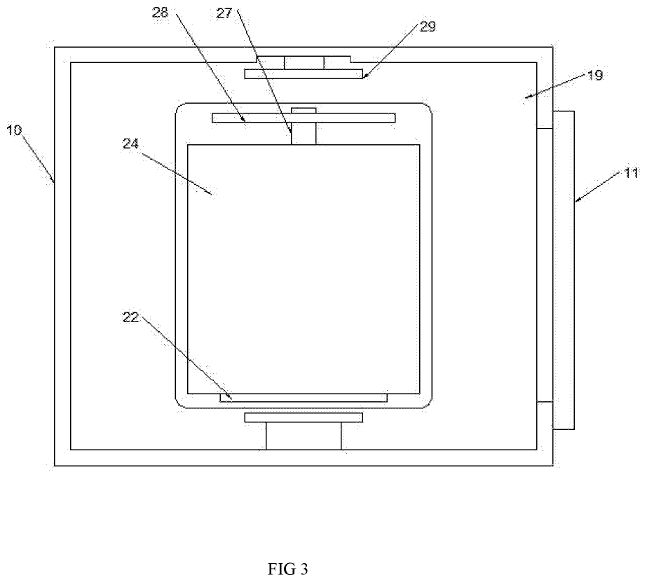

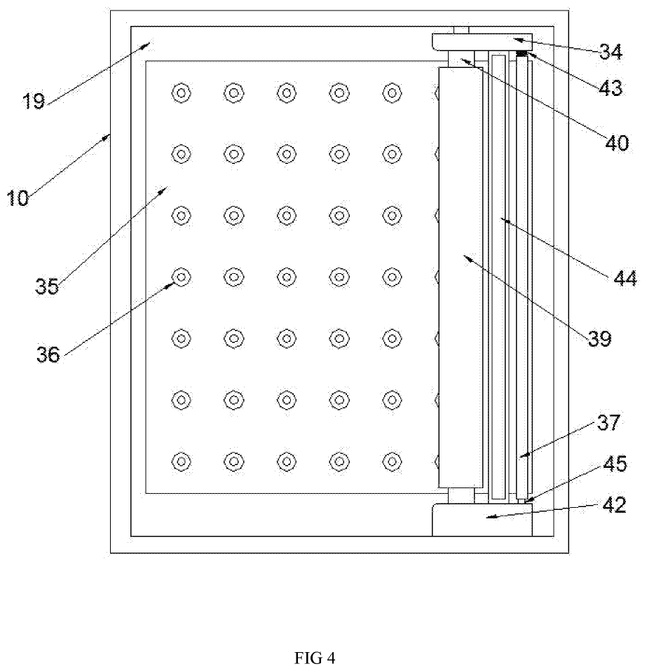

The present invention will be described in detail below with reference to FIGS. 1-4. For convenience of description, the orientation described below is defined as follows: the up-down-left-right-back direction described below is consistent with the up-down-left-right-left direction of the projection relationship of FIG.

A special sterilization cabinet for wooden cutting boards described in conjunction with FIGS. 1-4 mainly includes a sterilization cabinet 10. The sterilization cabinet 10 is provided with a sterilization chamber 19 that is opened forward, and the sterilization chamber 19 is provided with a support plate 25 The support plate 25 is provided with a clamping mechanism 70. The cutting plate 24 is placed on the left end surface of the support plate 25, and the cutting plate 24 is clamped by the clamping mechanism 70. The right side of the support plate 25 is provided. A pushing mechanism 73 that moves the support plate 25 to the left after the pushing mechanism 73 is activated. A first electric guide rail 33 is fixed in the top wall of the disinfection chamber 19, and an upper side is installed on the lower side of the first electric guide rail 33. A mounting block 34 is provided on the bottom wall of the disinfection chamber 19 with a lower mounting block 42 capable of sliding back and forth. A power motor 41 is provided in the lower mounting block 42. A rotary shaft 40 is dynamically connected to the upper side of the power motor 41. The upper end of the rotation shaft 40 is rotatably connected to the lower end surface of the upper mounting block 34. A grinding wheel 39 is fixed on the rotation shaft 40. A positioning sensor 38 is provided on the rear wall of the disinfection chamber 19. After the cutting board 24 moves a certain distance to the left, the pushing mechanism 73 stops, and the first electric The guide rail 33 is started to move the grinding wheel 39 forward, and the left end surface of the cutting board 24 is polished away to a certain thickness, so as to remove mold spots and knife marks. The disinfection chamber 19 is provided with a The cleaning mechanism 72 is turned on after the grinding is finished, and the grinding wheel 39 and the cutting board 24 are washed. The disinfection chamber 19 is provided with an ultraviolet disinfection mechanism 71. After the ultraviolet disinfection mechanism 71 is turned on, The cleaned cutting board 24 is sterilized by irradiation.

Advantageously, the cleaning mechanism 72 includes a fixing plate 35 fixedly disposed on the left wall of the disinfection chamber 19, a nozzle 36 is installed on the right end face of the fixing plate 35, and a water inlet pipe 32 extends into the disinfection chamber 19 and the The nozzle 36 is in communication, and a drain pipe 20 extending to the outside of the disinfection chamber 19 is provided in the bottom wall of the disinfection chamber 19.

Beneficially, the ultraviolet disinfection mechanism 71 includes ultraviolet lamps 29 symmetrically fixed up and down on the upper and lower inner walls of the disinfection cavity 19. A reflector 30 is provided on the left side of the ultraviolet lamp 29. After the ultraviolet lamp 29 is activated, the light passes through The reflecting mirror 30 reflects to the left end surface of the cutting board 24.

Beneficially, an electric push rod 31 is fixed on the rear wall of the disinfection cavity 19, and a connecting rod 23 is fixed on the front end of the push rod on the front side of the electric push rod 31, and a cover plate is symmetrically provided on the front surface of the connecting rod 23. 21. The cover plate 21 shields the reflecting surface of the reflecting mirror 30, thereby avoiding contamination of the reflecting surface of the reflecting mirror 30 during grinding and cleaning.

Beneficially, the pushing mechanism 73 includes a rotary motor 16 disposed in the right wall of the disinfection chamber 19, and a threaded rod 17 is dynamically connected to the left side of the rotary motor 16. The disinfection chamber 19 is provided with the screw thread. A threaded sleeve 18 to which the rod 17 is screwed. The left end of the threaded sleeve 18 is fixedly connected to the support plate 25. The threaded sleeve 18 is provided with a guide groove 15 which is opened upward. The disinfection chamber 19 is provided with The restriction block 26 is slidably installed in the guide groove 15. When the rotary electric machine 16 is started, the threaded sleeve 18 moves to the left under the restriction of the restriction block 26.

Beneficially, a second electric guide rail 14 is fixedly arranged on the inner wall of the disinfection chamber 19, and a middle rod 13 is installed on the left side of the second electric guide rail 14, and the middle rod 13 is fixedly connected to the restriction block 26. After the cutting board 24 is rinsed, the second electric guide rail 14 is activated to lift the restriction block 26 away from the guide groove 15, and the rotary motor 16 is activated to cause the threaded rod 17 to drive the threaded sleeve 18. Rotate to dry the cutting board 24.

Advantageously, the clamping mechanism 70 includes a lower clamping plate 22 fixedly disposed on the left end surface of the support plate 25, and a third end of the support plate 25 is fixedly provided on the upper side of the lower clamping plate 22 An electric guide rail 27, an upper clamping plate 28 is installed on the left side of the third electric guide rail 27, and the anvil plate 24 is placed between the lower clamping plate 22 and the upper clamping plate 28 to activate the first clamping plate 28; The three electric guide rails 27 lower the upper clamping plate 28 to clamp the anvil plate 24, and the length of the lower clamping plate 22 and the upper clamping plate 28 is less than the thickness of the anvil plate 24.

Beneficially, a dust suction mechanism 44 located on the rear side of the 39 is installed between the upper mounting block 34 and the lower mounting block 42, and the rotation between the upper mounting block 34 and the lower mounting block 42 is provided. A mounting shaft 45 is connected between the mounting shaft 45 and the top wall of the upper mounting block 34. A torsion spring 43 is fixed on the mounting shaft 45. A shielding plate 37 is fixed on the rear side of the dust collection mechanism 44.

Beneficially, a cabinet door 11 is hingedly installed on the front surface of the disinfection cabinet 10 for closing the opening on the front side of the disinfection chamber 19.

Beneficially, an indicator light 12 is installed on the front surface of the disinfection cabinet 10, and the indicator light 12 can light different colors during and after disinfection.

The working steps are as follows: open the cabinet door 11, place the lower end of the cutting board 24 on the upper end surface of the lower clamping plate 22, activate the third electric guide rail 27 to lower the upper clamping plate 28 and clamp the anvil plate 24, and start the rotary motor 16 to make the threaded rod 17 Rotation, the threaded sleeve 18 drives the support plate 25 to move to the left, and when the positioning sensor 38 senses that the anvil plate 24 moves to the left by a certain distance, the rotary motor 16 is turned off;

The power motor 41 is started to rotate the rotary shaft 40 to drive the grinding wheel 39, and the first electric guide rail 33 is started to move the grinding wheel 39 forward, and the left end surface of the cutting board 24 is polished to remove mold spots and knife marks. After the grinding is finished, the first The electric guide rail 33 is started to reset the backward movement of the grinding wheel 39;

The water inlet pipe 32 turns on the water supply, and the nozzle 36 sprays water to rinse the grinding wheel 39 and the cutting board 24;

After the flushing is completed, the rotary motor 16 is activated to reset the support plate 25 to the right. The second electric guide rail 14 is activated to cause the intermediate rod 13 to drive the restricting block 26 upward to disengage from the guide groove 15. The rotary motor 16 is activated and the threaded rod 17 is driven The threaded sleeve 18 rotates, so that the support plate 25 drives the anvil plate 24 to rotate and spin-dry;

After the spin-drying is completed, the second electric guide rail 14 is activated so that the limiting block 26 is inserted into the guide groove 15, and the electric push rod 31 is activated so that the connecting rod 23 drives the cover plate 21 to move backward without blocking the reflecting surface of the reflector 30, and the ultraviolet lamp 29 is turned on. The light is reflected by the reflector 30 to the left end surface of the cutting board 24 for disinfection. After the disinfection is completed, the indicator light 12 lights up. At this time, the cutting board 24 can be taken out.

The above embodiments are only for explaining the technical concept and characteristics of the present invention, and the purpose thereof is to enable those skilled in the art to understand and implement the content of the present invention, but not to limit the protection scope of the present invention. Any equivalent change or modification made according to the spirit and essence of the present invention should be covered by the protection scope of the present invention.

1. A special disinfection cabinet for wooden cutting boards includes a disinfection cabinet. The disinfection cabinet is provided with a disinfection cavity opening forward, and is characterized in that a support plate is provided in the disinfection cavity and a clamping mechanism is provided on the support plate. Placing the cutting board on the left end surface of the support plate, clamping the cutting board by the clamping mechanism, and a pushing mechanism is provided on the right side of the support plate, and the support plate is moved to the left after the pushing mechanism is activated, A first electric guide rail is fixed in the top wall of the disinfection chamber, an upper mounting block is installed on the lower side of the first electric guide rail, and a lower mounting block is provided on the bottom wall of the disinfection chamber, which can be slid back and forth. A power motor is provided in the upper side of the power motor, a rotary shaft is dynamically connected, the upper end of the rotary shaft is rotatably connected to the lower end surface of the upper mounting block, a grinding wheel is fixed on the rotary shaft, and the rear wall of the disinfection chamber A positioning sensor is provided. After the positioning sensor detects that the cutting board moves to the left for a certain distance, the pushing mechanism stops, the first electric guide rail starts to move the grinding wheel forward, and the left end surface of the cutting board is polished away. One To achieve the removal of mildew and knife marks, the disinfection chamber is provided with a cleaning mechanism on the left side of the grinding wheel. After grinding, the cleaning mechanism is turned on to rinse the grinding wheel and the cutting board. The disinfection chamber is provided with an ultraviolet disinfection mechanism. After the ultraviolet disinfection mechanism is turned on, the cleaned cutting board is irradiated and disinfected.

2. The special disinfection cabinet for wooden cutting boards according to claim 1, wherein the cleaning mechanism comprises a fixed plate fixedly arranged on the left wall of the disinfection chamber, a nozzle is installed on the right end face of the fixed plate, and the water inlet pipe extends It is in communication with the nozzle in the disinfection chamber, and a drain pipe extending out of the disinfection chamber is provided in the bottom wall of the disinfection chamber.

3. The special disinfection cabinet for wooden cutting boards according to claim 1, characterized in that the ultraviolet disinfection mechanism comprises ultraviolet lamps which are symmetrically fixed on the upper and lower inner walls of the disinfection chamber, and a reflector is provided on the left side of the ultraviolet lamp. After the ultraviolet lamp is activated, the light is reflected to the left end surface of the cutting board through the reflector.

4. The special disinfection cabinet for wooden cutting boards according to claim 3, characterized in that: an electric push rod is fixed on the rear wall of the disinfection chamber, and a connecting rod is fixed on the front end of the push rod on the front side of the electric push rod. A cover plate is symmetrically provided on the front surface of the connecting rod, and the cover plate shields the reflecting surface of the mirror, thereby avoiding contamination of the reflecting surface of the mirror during grinding and cleaning.

5. The special disinfection cabinet for wooden cutting boards according to claim 1, wherein the pushing mechanism comprises a rotary electric machine provided in the right wall of the disinfection cavity, and a threaded rod is connected to the left side of the rotary electric machine. The disinfection cavity is provided with a threaded sleeve threadedly connected to the threaded rod, the left end of the threaded sleeve is fixedly connected to the support plate, the threaded sleeve is provided with a guide slot opening upward, and the disinfection cavity A restriction block slidably installed in the guide groove is provided therein. When the rotary electric machine is started, the threaded sleeve moves to the left under the restriction of the restriction block.

6. The special disinfection cabinet for wooden cutting boards according to claim 5, wherein a second electric guide rail is fixed in the inner wall of the disinfection chamber, and a middle rod is installed on the left side of the second electric guide rail, and the middle rod It is fixedly connected to the restriction block. After the cutting board is washed, the second electric guide rail is activated to lift the restriction block away from the guide groove, and the rotary motor is activated to cause the threaded rod to drive the block. The threaded sleeve is rotated to dry the cutting board.

7. The special disinfection cabinet for wooden cutting boards according to claim 1, wherein the clamping mechanism comprises a lower clamping plate fixedly disposed on the left end surface of the support plate, and the left end surface of the support plate is fixedly disposed on the left end surface of the support plate. A third electric guide rail on the lower side of the lower clamping plate, an upper clamping plate is installed on the left side of the third electric guide rail, and the cutting board is placed between the lower clamping plate and the upper clamping plate Starting the third electric guide rail to lower the upper clamping plate to clamp the anvil plate, and the length of the lower clamping plate and the upper clamping plate is less than the thickness of the anvil plate.

8. The special disinfection cabinet for wooden cutting boards according to claim 1, characterized in that a dust suction mechanism at the rear side is installed between the upper mounting block and the lower mounting block, and the upper mounting block and A mounting shaft is rotatably provided between the lower mounting blocks, a torsion spring is connected between the mounting shaft and a top wall of the upper mounting block, and a shielding located at a rear side of the dust collecting mechanism is fixed on the mounting shaft. board.

9. The special disinfection cabinet for wooden cutting boards according to claim 1, characterized in that: a front door of the disinfection cabinet is hingedly installed with a cabinet door for closing the front opening of the disinfection chamber, and an indicator is installed on the front surface of the disinfection cabinet Light, the indicator light can light different colors during and after disinfection.
